(19) United States
(12) Patent Application Publication
(10) Pub. No.: US 2023/0062365 A1
(43) Pub. Date: Mar. 2, 2023

(54) EXCISION APPARATUS COMPRISING A HOUSING PROVIDED WITH A FIXATION PORTION

(71) Applicant: Dirk Carl Luc COEMAN, Kapellen (BE)

(72) Inventor: Dirk Carl Luc COEMAN, Kapellen (BE)

(73) Assignee: Dirk Carl Luc COEMAN, Kapellen (BE)

(21) Appl. No.: 17/799,965

(22) PCT Filed: Feb. 17, 2021
(86) PCT No.: PCT/EP2021/053887

§ 371 Date: Aug. 16, 2022

(30) Foreign Application Priority Data
Feb. 17, 2020 (NL) 2024926

(51) Int. Cl. A61B 18/14

(52) U.S. Cl. CPC A61B 18/1485; A61B 2018/00601

(57) ABSTRACT

An excision apparatus for removing cellular tissue includes a housing provided with a fixation portion. The fixation portion is configured to be arranged on cellular tissue such that a closed space is formed by the cellular tissue and an inner surface of the fixation portion. The fixation portion is configured to fixedly retain the cellular tissue near the inner surface by removal of air from the closed space via an air evacuating means. The apparatus further includes a cutting element moveably arranged in the housing such that the cutting element is movable between a retracted position and an extended position with respect to the fixation portion. The cutting element includes an electrode arranged at a distal end of the cutting element and is configured for cutting a section of cellular tissue retained by the fixation portion when the cutting element is in the extended position.

### FIELD OF INVENTION

The present invention relates to an excision apparatus for removing cellular tissue, said excision apparatus comprising a housing provided with a fixation portion.

### BACKGROUND

Electrosurgery tools use a current passing through tissue to cut, coagulate, desiccate, or fulgurate that tissue. The electrosurgical tool produces a high-frequency electric current and sends the current via an electrode through a patient's body. The use of electrosurgery tools produces electrosurgical smoke. The surgical smoke contains hazardous organic and inorganic compounds. In other words, the surgical smoke comprises hazardous aerosolized particles. In addition to the patient, these aerosolized particles are harmful to the medical personnel when inhaled. Moreover, the aerosolized particles may contain bacteria, viruses or viral DNA from the treated tissue. This may transmit pathogens to the surroundings.

Surgical masks do not adequately filter the hazardous components of a surgical smoke. Moreover, surgical masks often fit loosely and allow aerosolized particulates to bypass all filter functions of the surgical mask and to be inhaled.

### SUMMARY

It is an object of the present invention to provide an excision apparatus which eliminates or reduces the release of surgical smoke. It is a further object of the invention to provide an excision apparatus with improved handling.

The invention provides for this purpose an excision apparatus for removing cellular tissue, said excision apparatus comprising a housing provided with a fixation portion, wherein the fixation portion is configured to be arranged on cellular tissue such that a closed space is formed by the cellular tissue and an inner surface of the fixation portion. The fixation portion is further configured to fixedly retain the cellular tissue by removal of air from the closed space via an air evacuating means. The excision apparatus further comprises a cutting element moveably arranged in the housing such that the cutting element is movable between a retracted position and an extended position with respect to the fixation portion. The cutting element comprises an electrode arranged at a distal end of the cutting element and is configured for cutting a section of cellular tissue retained by the fixation portion when the cutting element is in the extended position. By removing the air from the closed space formed by the cellular tissue and the fixation portion, an air pressure in said closed space is reduced. In this way at least a partial vacuum is created. The partial vacuum forces the cellular tissue in to the fixation portion. In that manner the cellular tissue is retained in the fixation portion. In this way, the fixation portion substantially prevents the tissue from moving during a surgeon's action. This reduces the chance that erroneous slip ups of the surgeon occur while operating the excision apparatus. Additionally, the quality of cut is improved and the amount of tissue that is removed may be limited to the requisite amount. This improves overall patient recovery rates. An additional advantage is that when cutting the tissue the generated surgical smoke is contained in the chamber. This substantially reduces or even completely eliminates the dispersion of the generated smoke, thereby reducing the hazardous and pathogenic risk.

Preferably, the cutting element is translatable along an axis with respect to the housing, wherein the axis A corresponds to a longitudinal direction of the housing. Because the cutting element is translatable with respect to the housing, a depth of cut of the cutting element may be controlled in an efficient manner.

Preferably, the fixation portion comprises, at an open end thereof, at least one standing wall having an edge configured to come into contact with the cellular tissue and wherein, in the extended position, the cutting element does not extend beyond the edge of the fixation portion. Preferably the at least one standing wall comprises a circumferential wall and the edge has a substantially circular shape. More preferably, the fixation portion is substantially cup-shaped.

Preferably, the cutting element is translatable over a distance measured along the axis A of at least 3 mm, preferably at least 6 mm, more preferably 9 mm.

Preferably, the cutting element is rotatable along the axis A of the housing. In this way an operator may determine the cutting direction as well as the total size of cut in a simple manner. This allows for the accurate removal of only a selective section of tissue. In this manner the removal of an excess amount of tissue is avoided, which in turn improves the treatment of the patient and allows for a faster recovery of the patient.

Preferably, the cutting element is rotatable between a plurality of indicated cutting positions, wherein each indicated cutting position represents a respective section of tissue to be cut. In this manner a visual feedback is provided to the surgeon while operating the excision apparatus. Because the cutting element is rotatable between the plurality of indicated cutting positions, a required cutting orientation for removing the total portion of tissue to be cut can easily be maintained. In other words, when a surgeon examines a patient and thereafter thus knows the location of the tissue that requires to be removed, the surgeon may arrange the excision apparatus on the tissue and use the plurality of indicated cutting positions to remove only the required tissue corresponding to a respective cutting position. Because the tissue is fixedly retained in the fixation portion, the surgeon may rely on the plurality of indicated cutting positions to efficiently remove only the required portion of tissue. This improves histopathological analysis after removal of the tissue, and thus reduces a total examination time. Preferably, the plurality of indicated cutting positions comprises four quadrants (I, II, III, IV).

Preferably, at least the fixation portion is manufactured from a transparent material. By manufacturing at least the fixation portion from a transparent material an operator may continuously inspect the tissue fixedly retained in the fixation portion.

Preferably, the excision apparatus further comprises a guiding rod extending outwardly along the axis A, in the direction of a distal end of the housing, and configured to be inserted in a body cavity, such as a cervix of a female patient, for aligning the excision apparatus with the walls of the body cavity.

Preferably, the loop electrode is a bipolar electrode comprising a first and a second subelectrode. An advantage hereof is based on the insight that typical monopolar electrodes circulate a current from the active electrode through a plurality of layers of the body towards a surface before returning to a generator. This may cause iatrogenic burns or may interfere with electronic devices such as pacemakers etc. By using a bipolar electrode the risk of interference with other electronic devices is reduced or substantially eliminated. Moreover coagulation is more efficiently performed and burn damage to surrounding tissue is reduced. An additional advantage is based on the insight of the inventor that when using a bipolar electrode substantially less surgical smoke is generated due to the lower operating temperatures. This further reduces the hazardous and pathogenic risk.

Preferably, the electrode forms the first subelectrode and wherein guiding rod forms the second subelectrode.

Preferably, the electrode is a loop electrode.

Preferably, the loop electrode extends radially, seen in a projection on the axis, over a distance of at least 3 mm, preferably at least 5 mm, more preferably at least 10 mm.

Preferably, the housing comprises an elongate intermediate portion, wherein the fixation portion is arranged at a distal end of said elongate intermediate portion and is operably connected to said elongate intermediate portion such that air is removable from the closed space via the air evacuating means through the intermediate portion.

Preferably, the air evacuating means is formed by the elongate intermediate portion being spaced apart from and around the cutting element such that an air removal channel is formed and wherein the intermediate portion comprises an air evacuation interface being connectable to an air pump. More preferably, one or more further air removal channels are formed by the elongate intermediate portion.

Preferably, an actuating element is further comprised being connected to the cutting element, wherein the actuating element is arranged at the proximal end of the housing and is configured for moving the cutting element between the retracted position and the extended position and/or for rotating the cutting element along the axis A.

Preferably, the actuator element is arranged in an actuator housing section comprising an actuator housing wall. The actuator housing wall comprises a plurality of position protrusions which are configured to be engaged by a position limiting element of the actuator element. This allows for an improved handling of the excision apparatus, at least in view of steadiness, positional feedback and/or orientation feedback. Preferably, the plurality of position protrusions define a plurality axially oriented translation channels and tangentially oriented orientation channels.

Preferably, the position limiting element slidely engages the plurality of position protrusions such that the actuating element is moved and positioned with respect to the housing.

Preferably, the actuator housing wall may be provided on the outside thereof with indicators corresponding to the plurality axially oriented translation channels and tangentially oriented orientation channels.

Preferably an actuator housing section is provided at the proximal end of the housing and comprises a plurality of slots arranged side by side seen in a longitudinal direction along the axis A and the excision apparatus further comprises a position abutting element configured to prevent the cutting element from moving beyond the position abutting element, wherein the position abutting element is arrangeable in a slot of the plurality of slots. Because the slots are arranged side by side seen along the longitudinal direction, the cooperation between the position abutting element and the actuator housing section provided with a plurality of slots allows to quickly and easily set a cutting depth of the cutting element. More preferably, the plurality of slots form a through hole and the position abutting element comprises an abutment which is intended to extend through the slot when the position abutting element is arranged on the actuator housing section and is configured to prevent the cutting element from moving beyond the abutment. In this way, when moving the cutting element from the retracted position to the extended position, the cutting element will engage the abutment which consequently limits the movement of the cutting element.

Preferably, the position abutting element is clippable on the housing, preferably using a flexible or spring-loaded clip configured to engage the housing. This allows to easily set the cutting depth of the excision apparatus to a predetermined value by fastening the position abutting element, using the clip, to the housing, in particular the actuator housing section thereof.

Preferably, the fixation portion is configured to be releasably attachable to the distal end of the housing. A releasable fixation portion allows to remove the cut tissue fixedly retained in the fixation portion without further use of for example a forceps.

Preferably, the fixation portion is provided with one or more air grooves on the inner surface thereof, wherein the one or more air grooves extend over the inner surface. The air grooves substantially enlarge the suction area of the fixation portion. In this way tissue is more evenly retained in the fixation portion in an improved way.

Preferably, the fixation portion is provided with a plurality of graduation indicators, wherein the plurality of graduation indicators are evenly distributed seen along a circular arc on the fixation portion. Using a fixation portion with such graduation indicators provides the surgeon with visual feedback in a simple manner which reduces the risk for the patient and improves the patient's recovery after the intervention.

### BRIEF DESCRIPTION OF THE FIGURES

The accompanying drawings are used to illustrate presently preferred non-limiting exemplary embodiments of devices of the present invention. The above and other advantages of the features and objects of the invention will become more apparent and the invention will be better understood from the following detailed description when read in conjunction with the accompanying drawings, in which:

FIGS. 1A, and 1B illustrate a cross sectional view of an exemplary embodiment of an excision apparatus in a retracted and an extended position;

FIG. 2 illustrates a cross sectional view of a further exemplary embodiment of an excision apparatus;

FIGS. 3A and 3B illustrate a front view of an excision apparatus seen in a plane perpendicular to the longitudinal direction of the apparatus;

FIGS. 4A, 4B and 4C illustrate schematic views of an exemplary embodiment of an actuating element;

FIG. 5 illustrates a perspective view of an exemplary embodiment of an excision apparatus;

FIG. 6A illustrates a side view of the exemplary embodiment of FIG. 5;

FIGS. 6B and 6C illustrate enlarged portions of a distal end of the cutting element comprising an electrode according to exemplary embodiments;

FIG. 7 illustrates a cross sectional view B-B of the elongate intermediate portion indicated in FIG. 5 according to an exemplary embodiment;

FIGS. 8A and 8B illustrate a front view of a fixation portion according to preferred embodiments;

FIG. 9 illustrates a perspective view of a fixation portion according to an exemplary embodiment of an excision apparatus.

### DESCRIPTION OF EMBODIMENTS

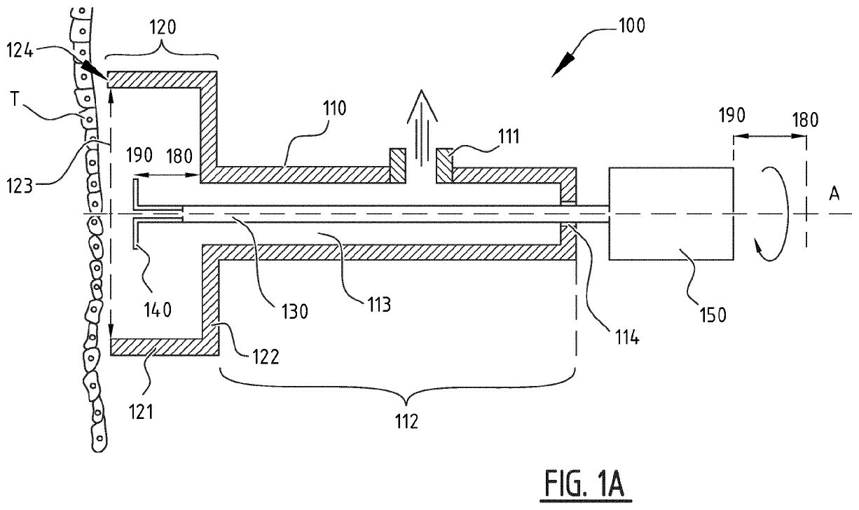

FIGS. 1A and 1B illustrate an exemplary embodiment of an excision apparatus 100 comprising a housing 110 provided with a fixation portion 120. The excision apparatus 100 is configured for removing cellular tissue T in, for example, a biopsy procedure. More generally the excision apparatus 100 may be used in any procedure for removal of tissue from any part of the body of a person or animal.

The fixation portion 120 is arranged at a distal end of the elongated housing 110 of the excision apparatus and is configured to be arranged on cellular tissue T. The illustrated fixation portion comprises at least one circumferential wall 121 extending from the base 122 over a distance in a direction along a longitudinal axis A of the housing 110. The at least one wall 121 is arranged such that it surrounds the axis A at a distance r. The distance r is preferably at least 5 mm, more preferably 7 mm, even more preferably at least 10 mm. The at least one wall 121 may be coupled to the housing 110 via the base 122 or may be integrally formed with the housing. Alternatively, the at least one wall 121 may be coupled directly to the housing using for example a coupling means. In this way the fixation portion 120 delimits a hollow core. The fixation portion comprises at least one standing wall having an edge 124 delimiting an open end 123 of the fixation portion 120. The edge 124 is configured to come into contact with cellular tissue. In other words, the edge 124 functions as a stopper. Preferably the edge defines a circular stop surface of the fixation portion 120. In such an embodiment the distance r may be considered to be the radius of the circular contact area. When the fixation portion 120 is arranged on cellular tissue a substantially closed space is formed by the cellular tissue T and the fixation portion 120. It will be clear that the fixation portion may take many shapes. Preferably the fixation portion 120 comprises a cup-like shape, such as a suction cup. In a further preferred embodiment the fixation portion is manufactured from a transparent material. The fixation portion 120 is further configured to fixedly retain the cellular tissue T by removal of air from the closed space via an air evacuating means 111. Preferably the fixation portion has a diameter of between 15 mm and 50 mm, more preferably between 20 mm and 40 mm depending on the intended purpose of the excision apparatus and the fixation portion thereof.

The excision apparatus 100 further comprises a cutting element 130 moveably arranged in the elongated housing 100. The cutting element 130 is movable between a retracted position 180 and an extended position 190 with respect to the fixation portion 120. In the retracted 180 the cutting element 130 is positioned outside of the closed space defined by the fixation portion 120. In the extended position 190 the cutting element 130 is positioned within the closed space defined by the fixation portion 120. The cutting element 130 comprises an electrode 140 arranged at a distal end thereof. The electrode 140 is configured for cutting a section of cellular tissue S retained by the fixation portion when the cutting element 120 is in the extended position as illustrated by FIG. 1B. While the cellular tissue S retained by the fixation portion 120 is shown in FIG. 1B as to fill the complete volume of the closed space, it is noted that the retained cellular tissue may only partially fill the volume of the closed space, in particular at least a volume near the walls of the fixation portion.

According to an embodiment the cutting element 130 is translatable along the axis A with respect to the housing 110, wherein the axis A is oriented according to a longitudinal direction of the housing 110. The cutting element 130 may be translatable over a distance measured along the axis A of at least 3 mm, preferably at least 6 mm, more preferably at least 9 mm. Translating the cutting element 130 over a distance measured along the axis A corresponds with setting or altering a cutting depth. In a preferred embodiment the cutting element 130 does not extend beyond the edge 124 of the fixation portion. The cutting element 130 may be rotatable around the axis A.

The housing 110 further comprises an intermediate portion 112, preferably an elongated intermediate portion 112, arranged between the fixation portion and coupled to an actuating element 150, wherein the fixation portion 120 is arranged at a distal end of the intermediate portion 112. The intermediate portion 112 may be operably connected to the fixation portion 120 such that air is removable from the closed space via the air evacuating means through the intermediate portion 112. In an exemplary embodiment an air evacuation tube such as a vacuum tube may be arranged in the intermediate portion 112. In a preferred embodiment the air evacuating means is formed by the intermediate portion 112 being spaced apart from and around the cutting element 130 such that an air removal channel 113 is formed. In other words, the intermediate portion comprises an at least partially hollow centre through which air may be evacuated.

The intermediate portion 112 may be provided with an air evacuation interface 111, wherein the air evacuation interface 111 is connectable to an air pump (not illustrated) or vacuum pump, the air pump or vacuum pump may be manually driveable or may comprise electric driving means such as an electromotor. The air pump or vacuum pump is typically readily available in any operating room. The air evacuation interface 111 may comprise a coupling interface which is compatible with a vacuum tube outlet and/or inlet. It is noted that while FIG. 1A illustrates that the air evacuation interface 111 is provided on the intermediate portion 112 it will be immediately apparent to the skilled person that the air evacuation interface may also be provided on the fixation portion 120 or any other portion of the housing 110.

The excision apparatus 100 may comprise an actuating element 150 being connected to the cutting element 130. The actuating element 150 may be arranged at a proximal end of the housing 110 and opposite of the fixation portion 120. The actuating element 150 is configured for moving the cutting element between the retracted position 200 and the extended position 190. Moreover, the actuating element 150 is configured for rotating the cutting element around the axis A. The actuating element 150 is connected to the cutting element 130.

The cutting element 130 may extend from the actuating element 150 to the fixation portion 120 through the intermediate portion 112. The cutting element 130 may extend from the proximally arranged actuating element 150 through a through hole in the proximal end of intermediate portion 112 of the housing. In an embodiment wherein the intermediate portion 112 comprises an air removal channel 113 a seal 114, such as an air seal, may be arranged. The air seal 114 is configured to limit air leakage through the through hole. In this way, the vacuum level in the air removal channel 113 may be substantially maintained.

FIG. 1B illustrates that the cutting element 130 is positioned in the extended position 190. A surgeon may move the cutting element from the retracted position 180 to the extended position 190. By moving the cutting element 130 the electrode 140 cuts the section of tissue S as illustrated in FIG. 1B. Optionally the surgeon may rotate the cutting element 130. More in particular, by moving the cutting element 130 from the retracted position 180 to the extended position 190, the electrode 140 is brought into contact with the tissue S as the electrode 140 is inserted in the closed space defined by the fixation portion 120. Preferably the electrode 140 is inserted in the closed space via a top portion of the fixation portion 120, for example via a top wall of the fixation portion.

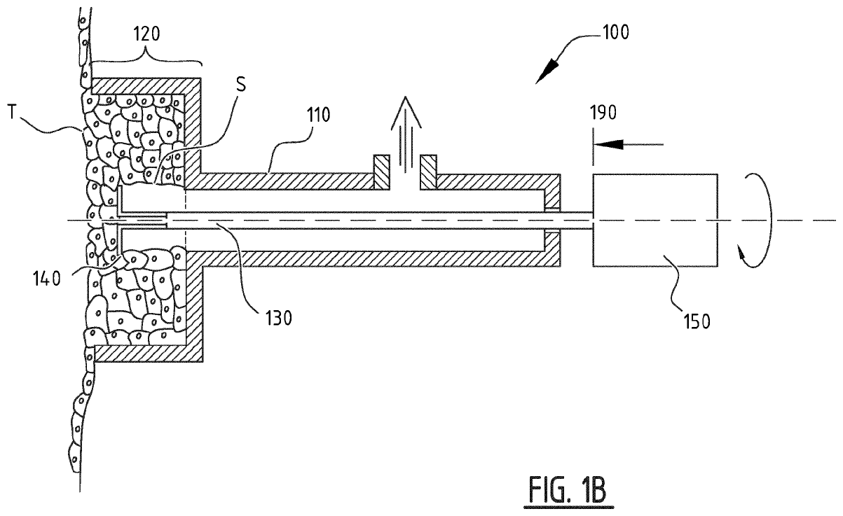

FIG. 2 illustrates a further embodiment of the excision apparatus 100. Similar or identical parts have been indicated with the same reference numerals as in FIGS. 1A and 1B, and the description given above for FIGS. 1A and 1B also applies for the components of FIG. 2. In the embodiment of FIG. 2 the excision apparatus is in particular configured for endo- and exocervical cone biopsies. The excision apparatus therefore comprises a guiding rod 200. The guiding rod 200 is configured for insertion in a cervix of a female patient for aligning the excision apparatus 110 with the cervix. The guiding rod 200 is fixed to the housing 110 at a first end 201 of the guiding rod and extends along the axis A of the housing 110 in the direction of the fixation portion 120 to a second end 202 thereof. In this way the alignment of the excision apparatus 100 with the cervix is improved and surgical imprecisions are substantially eliminated. The guiding rod 200 preferably extends beyond the fixation portion 120. By extending beyond the fixation portion, the guiding rod 200 further improves the alignment of the excision apparatus.

In a preferred embodiment cutting element 130 comprises a bipolar electrode comprising an active electrode 141 and a return electrode 142. The electrode 140 may be a loop electrode. The guiding rod may function as the return electrode 142 of the bipolar electrode. Optionally, the electrode 140 may comprise the active electrode and the return electrode, arranged opposite of each other. In the preferred embodiment of FIG. 2 the electrode is a loop electrode extending over a distance of at least 3 mm, preferably at least 5 mm, more preferably at least 10 mm.

FIG. 2 illustrates in particular that the axis of the cutting element 130 is arranged parallel to the guiding rod 200 such that a cutting element 130 is always correctly aligned.

The cutting element 130 may be operably connected to a power source 160 configured for providing power to the electrode. A first and second conductor (not illustrated) may be provided, wherein the first conductor electrically connects the active electrode to the power source 160 and the second conductor electrically connects the return electrode to the power source 160. In an embodiment where the guiding rod 200 comprises the return electrode of the bipolar electrode the second conductor is electrically connected to the guiding rod 200.

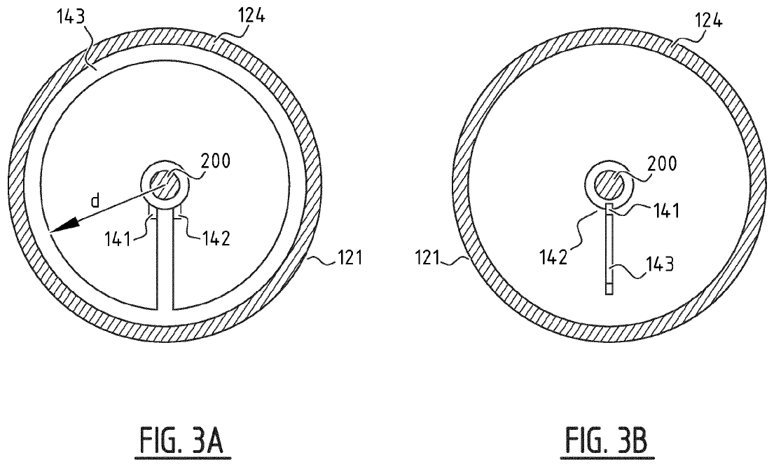

FIG. 2 further illustrates that the housing 110 comprises an actuator housing section 115 configured to house and/or engage with the actuating element 150. The actuator housing section 115 and further embodiments of the actuating element 150 will be discussed in detail in FIG. 4A, 4B, 4C. FIG. 2 further illustrates that the actuator housing section 115 comprises an actuator housing wall 116 extending in the proximal direction of the housing along the axis A. The actuator housing wall 116 delimits an at least partially hollow core configured to house at least a portion of the actuating element 150. The actuator housing wall 116 may comprise a plurality of position protrusions 117. Embodiments of the actuator housing section 115 will be further elaborated with respect to FIGS. 4A, 4B, 4C.

According to a possible embodiment a three-way valve 170 may be arranged between the air evacuating interface 111 and a vacuum pump 210, in for example a vacuum tube. The three-way valve comprises three positions, wherein a first position fluidly connects the vacuum pomp 210 with the air evacuating interface 111 such that air is removed from the closed space and the fixation portion fixedly retains tissue. In a second position the three-way valve 170 is configured to fluidly shut the closed space off such that a vacuum therein is maintained. In a third position the three-way valve 170 is configured to fluidly connect the closed space with a container such that substantially no polluted air may reach the vacuum pump.

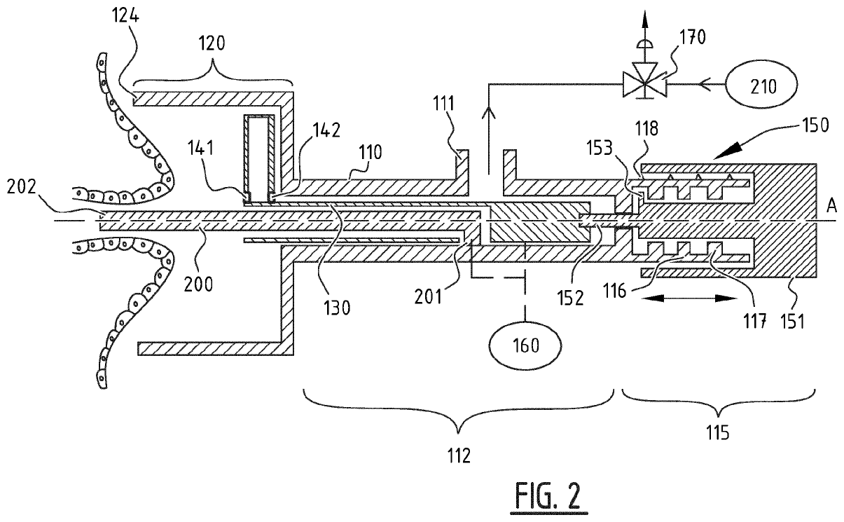

FIGS. 3A and 3B illustrate a front view of an excision apparatus, especially the cutting element and guiding rod thereof, seen in a plane perpendicular to the longitudinal axis of the apparatus. FIGS. 3A and 3B illustrate in particular alternative embodiments of the electrodes 140 illustrated in FIGS. 1A, 1B and 2. The electrodes 140 may be monopolar or bipolar. In the context of the application monopolar electrodes are cutting elements comprising a single active electrode and an inactive return electrode. The inactive electrode may for example be the operating table. In other words, a patient's body functions as the return electrode. Bipolar electrodes are cutting elements comprising an active electrode and a return electrode. In other words, bipolar electrodes comprise a first subelectrode and a second subelectrode. Contrary to monopolar electrodes the first and second subelectrode are both situated at or in close proximity of the excision location. An active electrode may also be referred to as a first subelectrode and a return electrode may also be referred to as a second subelectrode. The return electrode may also be an active electrode.

FIG. 3A illustrates an electrode wire 143 that may extend from the first subelectrode 141 to the second subelectrode 142 to form a loop in a plane substantially perpendicular to the axis A. While FIG. 3A illustrates that the second subelectrode is arranged in parallel to the first subelectrode 141, it will be clear that the guiding rod 200 may also form the second subelectrode as will be explained here below. A portion of the loop may be arranged at a radial distance d of the axis A. The electrode wire 143 is configured such that when the cutting element 130 is translated along the axis A, tissue is cut along a plane parallel to the axis A. This may be a circular loop, a semi-circular loop, a square loop, etc. The distance may be in the range of 4-11 mm. In a preferred embodiment the electrode 141 is the active electrode and the guiding rod 200 acts as the return electrode of a bipolar electrode. This results in a clean cut which allows the patient to recover more quickly. Moreover, this allows to easily remove the cut tissue is from the cutting section.

FIG. 3B illustrates a front view of the electrode of the embodiment of FIG. 2. The electrode 140 comprises a first subelectrode 141 and a second subelectrode arranged at a distance from each other measured along the axis A (as shown in FIG. 2) and are electrically connected by the electrode wire 143. The electrode wire 143 may form a loop wherein the electrode wire 143 extends radially from the axis A to the at least one wall 121 of the fixation portion and back. In this way the electrode will cut tissue along a radial line of the axis when the cutting element is translated along the axis A. When the cutting element is rotated the electrode wire 143 arranged in a loop will cut tissue in a tangential direction of the rotation around the axis A. This may be used to only cut a select portion of tissue, of for example tissue of the cervix such as part of the endocervical canal during a cone biopsy.

FIGS. 4A, 4B, 4C schematically illustrate a preferred embodiment of the actuating element 150 which is preferably arranged in the actuator housing section 115 of the housing 110. Similar or identical parts have been indicated with the same reference numerals as in FIG. 2, and the description given above for FIGS. 1A, 1B and 2 also applies for the components of FIGS. 4A, 4B, 4C.

FIG. 4A illustrates in particular a cut view along the axis A of the actuating element 150 arranged in the actuator housing section 115. The actuator housing section 115 comprises an actuator housing wall 116 extending in the proximal direction of the housing along the axis A. The actuator housing wall 116 delimits an at least partially hollow core configured to house at least a portion of the actuating element 150. The actuator housing wall 116 may comprise a plurality of position protrusions 117, wherein each of the position protrusions 117 defines a unique position of the actuating element 150 with respect to the housing 110.

As illustrated in FIG. 4B, which shows an unfolded view of the interior of the actuator housing section 115, the position protrusions 117 are arranged in a grid pattern. The position protrusions 117 are in particular distributed in a plurality of cutting positions I, II, III, IV and in a plurality of rows A, B, C wherein the rows are each arranged at a longitudinal distance of each other, as seen along the axis A. The position protrusions are preferably distributed in four cutting positions. The four cutting positions are evenly distributed along a circumference of a circle seen in a projection on a plane perpendicular to the axis A. The four cutting positions respectively represent cutting angles from 0°-90°, 90°-180°, 180°-270° and 270°-360°. It will be clear that more than four cutting positions may be provided, for example six or eight cutting protrusions. In other words, the cutting element 130 is rotatable between a plurality of indicated cutting positions wherein each indicated cutting position corresponds with a respective section of tissue. In a preferred embodiment the plurality of indicated cutting positions comprises four quadrants. It will be clear to the skilled person that a surgeon may rotate the actuating element from a first rotation position to a second rotational position located in any of the cutting position at any angle, for example from 0° in the first cutting position I to 127° located in the second cutting position II. The actuator housing section may be provided with a protractor indicating the respective angles and cutting positions. Four position protrusions 117AI, 117AII, 117AIII and 117AIV are preferably arranged in a first row A, four position protrusions 117BI, 117BII, 117BIII and 117BIV in a second row B and four position protrusions 117CI, 117CII, 117CIII and 117CIV in a second row B. The plurality of position protrusions define a plurality axially oriented translation channels 118 and tangentially oriented orientation channels 119. The position protrusions may be arranged such that the translation channels define a cutting depth of at least 3 mm, preferably at least 5 mm, more preferably at least 10 mm. The translation channels 118 and orientation channels 119 are configured to guide a position limiting element 153 arranged on the actuating element 150 to an intended position. The position limiting element 153 slidely engages the position protrusions such that the actuating element 150, and by extension the cutting element, is moved and positioned with respect to the housing 110. Moreover, the actuator housing wall 116 may be provided on the outside thereof with indicators 220a, 220b, 220c corresponding to the respective rows A, B, C and optionally cutting positions I, II, III, IV such that the surgeon has a visual feedback of the cutting distance as well as orientation. In the retracted position the actuating element 150 may for example be positioned such that the position limiting element 153 engages the position protrusion 117AI first row A in cutting position I thereby determining a starting position of the cutting element. Based on a prior evaluation of the tissue the surgeon knows that two section of tissue must be removed at a depth of for example 4 mm. Based on the visual feedback provided by the actuating element 150 in conjunction with the indicators the surgeon knows to translate the actuating element to row B and rotate to cutting position III. With this excision apparatus physically difficult to reach biopsies as well as tissue removal operations where the visibility is limited due to a limited amount space may be performed in an effective and simple way. Additionally, the inventive insight hereof is further based on the fact that endo- and exocervical cone biopsies are intrinsically difficult and dangerous for the patient, such operations may cause infertility and other unwanted problems. In particular with female patients situated in the age group: teens to middle aged such operations are therefore often not performed. Because the excision apparatus fixedly retains the tissue and because the actuating element in conjunction with the actuator housing section provides the surgeon with the improved visual feedback such interventions may yet be performed in safety and with a high rate of success.

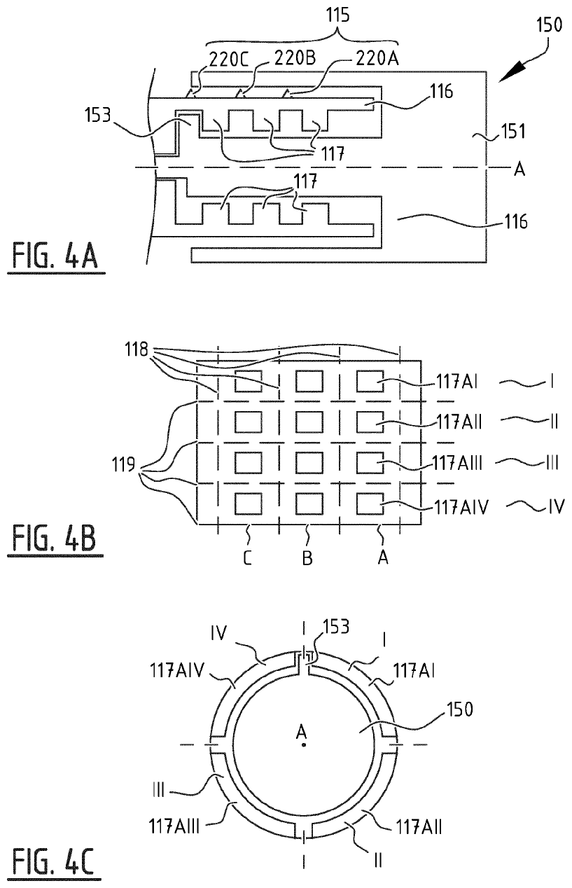

FIG. 5 illustrates a perspective view of an excision apparatus 100 according to a preferred embodiment.

In the illustrated exemplary embodiment the excision apparatus 100 comprises a housing 110. The housing 110 comprises an elongate intermediate portion 112. The housing 110 further comprises a handling portion 230. The handling portion 230 is provided in such a way that a user may easily operate the excision apparatus 100. In the illustrated preferred embodiment, the handling portion 230 is angularly orientated with respect to the elongate intermediate portion 112. In other words, the handling portion 230 and the elongate intermediate portion form an angle with respect to each other. Such an angular orientation of the handling portion 230 allows a surgeon to hold and manoeuver the excision apparatus 100 without blocking the view with respect to the area of the intervention.

The handling portion 230 may be integrally formed with the intermediate portion 112, i.e. the handling portion and the intermediate portion may be formed from a single piece. In this way, the integrally formed handling portion 230 and intermediate portion 112 form a robust housing 110 which allows the user to accurately position the excision apparatus. Alternatively, the handling portion 230 may be interlockingly connected to the intermediate portion 112 in a releasable way. To interlockingly connect the intermediate portion 112 to the handling portion 230 in preferably a releasable way, a connection interface 231a may be provided. The connection interface 231a of the handling portion 230 is configured to receive a corresponding connection interface 231b provided by the intermediate portion 112, or vice versa. The connection interface 231a of the handling portion 230 is for example provided with an internal screw thread in which a corresponding external screw thread which is provided on the connection interface 231b may be threaded, or vice versa. According to a further example the connection interfaces 231a, 231b may each form a respective part of a ratchet-like locking mechanism wherein a male portion is provided on one of the connection interfaces and is configured to interlockingly engage a female portion provided on the other corresponding connection, interface. This allows to connect and disconnect the handling portion 230 to and from the intermediate portion 112. Having a releasable intermediate portion 112 and/or handling portion 230 allows to produce the elongate intermediate portion 112 and the handling portion 230 independently from each other. This simplifies the production process of such an excision apparatus. Also, the intermediate portion 112 may for example be a disposable item and the handling portion 230 may be a reusable item.

As is apparent from FIG. 5 the air evacuation channel 113 further extends from the elongate intermediate portion 112 to an end of the handling portion 230. As has been explained with respect to FIG. 1A, the air evacuation interface 111 may be provided in a plurality of positions. In the illustrated exemplary embodiment of FIG. 5 the air evacuation interface 111 is arranged near an end of the handling portion 230. In this way, air evacuation means such as a vacuum tube and its connection elements are positioned relatively far from the intervention area such that they are “out of the way” and do not inhibit the actions of the surgeon or block the view of said surgeon.

The handling portion 230 may be provided with power coupling means 232 configured to provide power from a power source (not shown) to the electrode. The power coupling means 232 is for this reason provided with an electrical contact (not shown) such that power is transferred from the power source to the electrode even while the electrode is moving. The electrical contact may for example be a carbon brush, brush contact or a slip ring.

The illustrated excision apparatus 100 further comprises a fixation portion 120. The fixation portion 120 is arranged at a distal end of the elongate intermediate portion 112 of the housing 110. In the preferred embodiment of FIG. 5, the fixation portion 120 is transparent such that the surgeon has a visual feedback regarding the area of intervention and may safely perform the intervention, even when the fixation portion fixes a cellular tissue. In this way, the quality of the intervention as well as the patient's recovery is further improved.

The fixation portion 120 is, according to the illustrated preferred embodiment, releasable. A releasable fixation portion 120 allows to remove the cut tissue fixedly retained in the fixation portion 120 without further use of for example a forceps. More in particular, once the tissue has been cut, the fixation portion 120 holds the tissue. The removable fixation portion then holds the cut tissue. In this way, the removable fixation portion 120 beneficially serves a dual purpose. On the one hand, the fixation portion allows to perform the surgical intervention safely and with a substantially reduced risk of medical complication post-intervention. On the other hand, the fixation portion 120 is useable as a holder for the removed tissue. Contamination of the cut tissue is thus substantially reduced or avoided entirely. This improves histopathological analysis after removal of the tissue, and thus reduces a total examination time.

According to a preferred embodiment (not shown) the fixation portion 120 is rotatable with respect to the housing 110, in particular the elongate intermediate portion 112. Also the elongate intermediate portion 112 may be rotatable with respect to the handling portion 120. Such a preferred embodiment allows the surgeon to set the initial cutting angle while holding excision apparatus in a comfortable manner. The advantage thereof is based on the insight that the surgeon would otherwise have to rotate the entire excision apparatus to set the initial cutting angle. As has been elaborated here above, in the context of endocone or exocone interventions operating space is strictly limited and encumbers the surgeon. Having a rotatable fixation portion 120 and/or intermediate portion 112 provides the surgeon with a more ergonomic tool and simplifies the intervention.

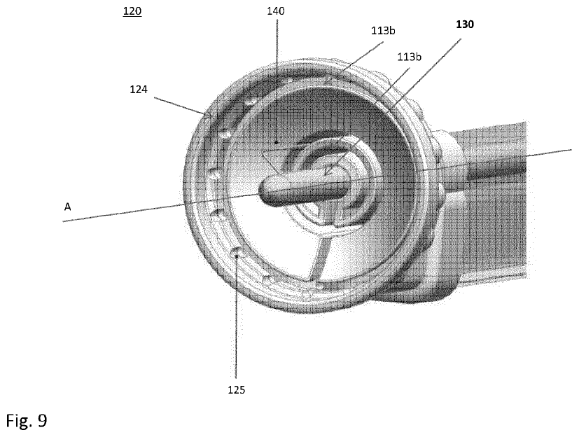

The fixation portion 120 is preferably provided with a plurality of graduation indicators 125 as illustrated in FIG. 9. Preferably, the plurality of graduation indicators 125 are evenly distributed along a circular arc line of the fixation portion 120. The circumferential line is preferably circular. The plurality of graduation indicators 125 will be further elaborated with respect to FIG. 9.

FIG. 5 illustrates a preferred embodiment of the actuating element 150 and the actuator housing section 115. The actuator housing section 115 is comprised in the housing 110 of the excision apparatus. In the illustrated embodiment the actuator housing section 115 comprises a plurality of slots 240. Each of the slots 240 is configured to receive a position abutting element 250. The slots 240 are arranged side by side, seen in a longitudinal direction along the axis A. In other words the slots are space apart a distance, seen along the axis A. The slots 240 extend from the outside of the actuator housing wall 116 to the inside. In other words, a through hole is formed by each slot 240.

The position abutting element 250 is arrangeable in any slot of the plurality of slots 240. The position abutting element 250 comprises a protrusion which is configured to extend through the respective slot to the inside of the actuator housing wall 116. The protrusion forms an abutment which prevents the cutting element 130 from moving beyond the abutment, seen along the axis A. The position abutting element 250 thus allows to set or limit the cutting depth of the cutting element 130. More in particular by arranging the position abutting element 250 such that the protrusion extends through a slot, the movement of the position abutting element 250 is limited by the actuator housing wall 116 delimiting the opening of the slots 240. Further, the abutment formed by the protrusion on the inside of the actuator housing wall 116 limits the movement of the cutting element 130. The cooperation between the position abutting element 250 and the actuator housing section 115 provided with a plurality of slots 240 thus allows to quickly and easily set a cutting depth of the cutting element 130. The slots 240 may be provided at a predetermined pitch distance from each other, meaning that the distance between neighbouring slots is the same. A slot 240 may for example be provided every 2 mm, seen along the longitudinal axis A. Alternatively, the slots 240 may be arranged at predetermined distances from each other which may be mutually different. For example, a distance of 4 mm may be present between the first and second slot, and a distance of 2 mm may be present between the second and third slot. It will be clear that the position abutting element 240 of the above preferred embodiment functions similarly to the cooperation of the position limiting element 153 in the translation channels 118 as elaborated with respect to FIGS. 4A and 4B. The position abutting element 250 is a preferred embodiment which is structurally less complex to produce than the embodiment of FIGS. 4a and 4B, while providing the surgeon with an accurate gauge of cutting depth in a simple manner. In a preferred embodiment which is particularly beneficial for exocone and/or endocone interventions, the slots are arranged in such a manner to allow the selection of cutting depths of around 4 mm, 10 mm, 20 mm, and 30 mm.

The position abutting element 250 can be attached to the housing 110 in a removable manner Preferably, the position abutting element 250 is clippable on to the housing of the excision apparatus. In other words, the position abutting element 250 comprises a flexible or spring-loaded clip which holds the position abutting element 251 and the excision apparatus 100 together. This allows to easily set the cutting depth to a predetermined value by fastening the position abutting element 250, using the clip, to the housing 110, in particular the actuator housing section 115 thereof.

According to a non-illustrated preferred embodiment, the cutting element or the actuating element 150 is operable by means of a hand- or finger operated trigger arranged on the handling portion. A hand operated trigger may for example be configured to rotate the cutting element 130, where squeezing the hand trigger over a certain distance would result in the cutting element rotating over a corresponding amount of degrees with respect to the squeezing distance. Alternatively or in combination, trigger may be configured to rotate the cutting element 130, optionally using the actuating element 150, a predetermined amount of degrees, for example 360°. This simplifies the intervention for the surgeon. Alternatively or in combination a foot operated pedal may be provided with similar functionality. Said hand operated trigger or foot pedal may also be configured to operate the vacuum pump or the three-way valve thus further increasing the operating possibilities.

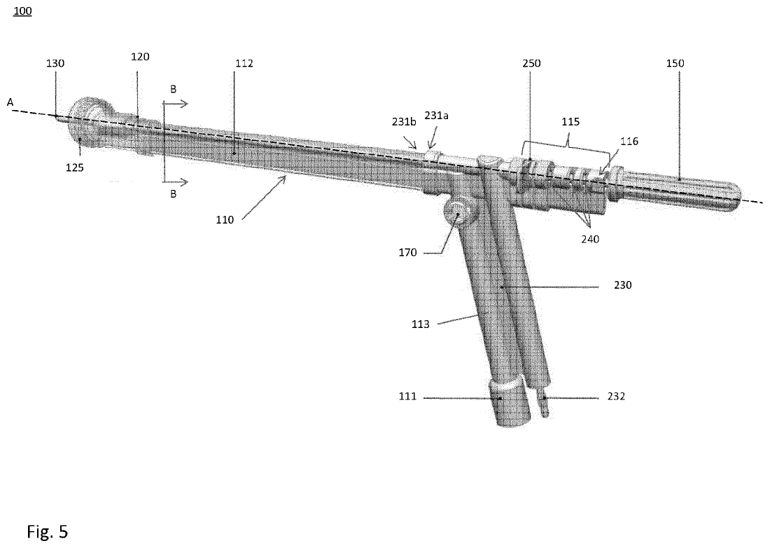

FIG. 6A is a side view of a preferred embodiment of the excision apparatus 100 illustrated in FIG. 5 where the cutting element 130 is illustrated separately from the housing 110.

FIG. 6A illustrates that the cutting element 130 may be inserted into the housing 112 at a proximal end P of the housing. The cutting element 130 is in particular insertable into housing 112 by sliding the cutting element 130 into the housing 112 along the axis A.

FIG. 6A also illustrates that according to the illustrated preferred embodiment the cutting element 130 comprises an actuating element 150 and an electrode 140. The actuating element 150 is arranged at a proximal end P of the cutting element 130. The electrode 140 is arranged near the distal end D of the cutting element 130. The cutting element 130 comprises an elongate shaft 131 which extends between the distal and proximal ends. The cutting element 130 comprises an electrical conductor 132 configured to transfer power from a power source (not shown) to the electrode 140. The electrical conductor 132 extends through the elongate shaft 131 from a power receiving section to the electrode 140. The power receiving section 133 is arranged and dimensioned such that when the cutting element 130 is arranged in the housing 110, the power receiving section 133 may electrically connect to, for example, the power coupling means 232. The power receiving section may also directly receive power from the power source (not shown). The power receiving section 133 may electrically connect to the power coupling means 232 in both the extended and retracted position of the cutting element 130, or any position therein between. In this way it is guaranteed that the electrode 140 is functional when the power source is turned on.

According to a preferred embodiment the elongate shaft 131 at least partially forms the electrical conductor 132, for example, the elongate shaft 131 may be partially fabricated from an electrically conductive material such as stainless steel. This advantageously provides the excision apparatus with a sturdy cutting element 130 whilst simultaneously providing an electrical conductor to provide power to the electrode 140. Preferably, the elongate shaft is coated or provided with a surface layer. The coating or surface layer is electrically insulating to prevent unwanted currents running through the body of the patient when the surface of the elongate shaft touches tissue. Also, unwanted cauterization or cuts are prevented because only the electrode 140 is able to cut or cauterize tissue in such an embodiment. Alternatively, the elongate shaft 131 may be hollow. The hollow shaft 131 may house the electrical conductor.

FIGS. 6B and 6C illustrate different preferred embodiments of the electrode 140 arranged at a distal end of the cutting element 130 as indicated in FIG. 6A.

FIG. 6B illustrates an electrode 140 which is optimally shaped for exocone interventions, for example to cut away tissue located outside the cavity of the cervix and near the external orifice of the cervix. The illustrated electrode 140 comprises a transversal portion extending away from the cutting element 130 which is relatively long, and a longitudinal portion extending along the cutting element which is relatively short. Preferably the transversal portion is curved to define a cutting curve at a distal end of the electrode as illustrated in FIG. 6B. The transversal portion is substantially perpendicular to the longitudinal axis A. Preferably the length of the transversal portion is between 5 mm and 20 mm, more preferably between 7 mm and 15 mm The longitudinal portion is substantially parallel to the longitudinal axis A. Preferably the length of the longitudinal portion is between 1 mm and 10 mm, more preferably between 2 mm and 8 mm. FIG. 6C illustrates an electrode which is optimally shaped for endocone interventions, for example to cut away tissue located inside the cavity of the cervix and beyond the external orifice of the cervix. The illustrated electrode 140 comprises a transversal portion extending away from the cutting element 130 which is relatively short, and a longitudinal portion extending along the cutting element which is relatively long. The transversal portion is substantially perpendicular to the longitudinal axis A. Preferably the length of the transversal portion is between 1 mm and 4 mm. The longitudinal portion is substantially parallel to the longitudinal axis A. Preferably the length of the longitudinal portion is between 10 mm and 50 mm, more preferably between 15 mm and 45 mm, most preferably between 20 mm and 40 mm. The illustrated electrodes 140 in FIGS. 6B and 6C are monopolar. Monopolar electrodes are more easily constructable and reduce the production costs of the excision apparatus. In combination with the fixation portion 120 the use of such monopolar electrodes are of no medical threat to the acting surgeon or patient since the smoke generated by cutting tissue is evacuated using the air evacuation means.

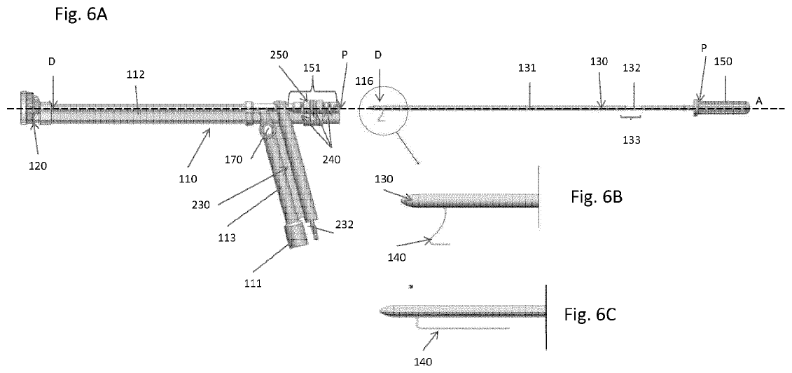

FIG. 7 illustrates a front view of the cross-section B-B indicated in FIG. 5. FIG. 7 illustrates in particular a cross sectional view of a preferred embodiment of an elongate intermediate portion 112 of the housing 110 of the excision apparatus 100. The intermediate portion 112 comprises a cutting element housing section 260. The cutting element housing section 260 is designed to accommodate the elongate shaft 131. The cutting element housing section 260 is designed such that the distance between the cutting element housing section 260 and the elongate shaft 131 is minimal, for example smaller than 5 mm, preferably smaller than 2 mm. In this way, cutting deviations are reduced because the freedom of movement of the cutting element 130 is substantially limited. In other words, the movement of the cutting element 130 is allowed only in the direction of the longitudinal axis A or in a rotational manner around said axis A. For improved rotational guidance of the cutting element 130, the inner circumference of the cutting element housing section 260 may be circularly shaped.

The cutting element housing section 260 is connected to an electrode housing section 261. The electrode housing section 261 extends radially from the cutting element housing section 260 in an outward direction. The electrode housing section 261 is configured to allow passage of the electrode 140, for example the electrode 140 illustrated in FIGS. 6B and 6C. The cutting element housing section 261 is preferably narrow such that the overall dimensions of the excision apparatus may be kept to a minimum. The radial distance over which the electrode housing section 261 extends is preferably at least 3 mm, preferably at least 5 mm, more preferably at least 10 mm. The combination of cutting element housing section 260 and electrode housing section 261 allows to easily insert the cutting element 130 illustrated in for example FIGS. 6A, 6B, 6C, in the excision apparatus. This allows the surgeon to easily exchange cutting elements or to load the excision apparatus before initializing the intervention.

In addition to the single air removal channel 113 formed by the housing 110 in FIGS. 1A and 1B, the intermediate portion 112 as illustrated in FIG. 7 comprises a plurality of further air removal channels 113. Put differently, one or more further air removal channels 113 are comprised in the intermediate portion 112. In the illustrated example two further air removal channels 113 are provided. The further air removal channels 113 improve air removal from the fixation portion 120. Also, the further air removal channels 113 are advantageously redundant. More in particular, when tissue, bodily slimes or fluids block one or more of the air removal channels 113, the fixation portion may continue to retain the cellular tissue as long as at least one of the air removal channels 113 remain at least partially open.

In the illustrated exemplary embodiment, the further air removal channels 113 are adjacently arranged with respect to the electrode housing section 261. Arranging a plurality of further air removal channels 113 has the advantage that the housing wall forming each of the further air removal channels 113 provides structural strength to excision apparatus. Thus the excision apparatus is more robust and stiff which allows to surgeon to more accurately perform the intervention.

In the illustrated embodiment the further air removal channels 113 are parallel to the electrode housing section 261 and comprise a substantially similar width. The elongated cross sectional shape of the air removal channels 113 allows to have a maximum possible air flow while maintaining the width of the overall housing of the excision apparatus to a minimum. It will be clear to those skilled in the art that more than two air removal channels 113 may be provided or that the shape thereof may differ. The air removal channel may for example be provided such that they surround the cutting element housing section 260 and the electrode housing section 261. Optionally, also the cutting element housing section 260 and electrode housing section 261 is connected to a vacuum pump.

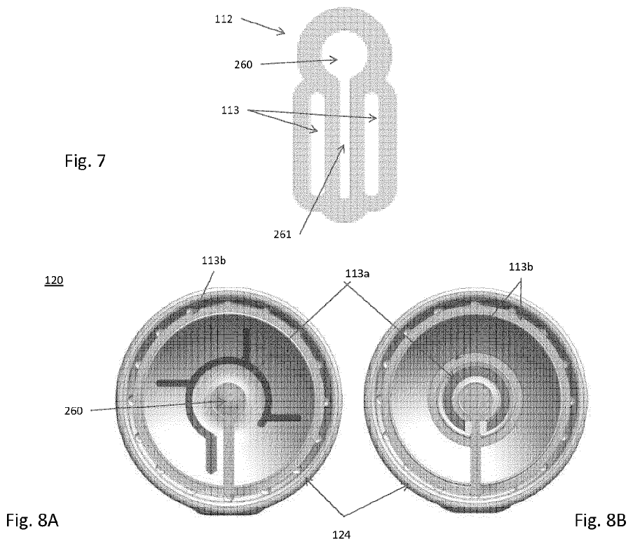

FIGS. 8A and 8B illustrate a front view of a fixation portion 120 according to preferred embodiments. In the illustrated embodiments an inner surface of the wall of the fixation portion 120 is provided with one or more air grooves 113a, 113b. The air grooves 113a, 113b are fluidly connectable to the air removal channel 113 and/or the cutting element housing section 260 and electrode housing section 261. The air grooves 113a, 113b improve the level of vacuum in the fixation portion 120 during use because the air grooves 113a, 113b distribute the vacuum over a larger area in the fixation portion 120. Put differently, the air grooves are designed to provide a large air inlet to the air removal channels which improves the level of vacuum in the fixation portion 120 during use of the excision apparatus. The larger air inlet substantially avoids a situation where cellular tissue blocks the air inlet to the air removal channels which would negatively influence the retaining of the tissue. The air grooves allow tissue to be more evenly retained in the fixation portion, thereby improving overall functionality of the excision apparatus in terms of tissue fixation, cutting accuracy, visibility, and/or durability.

In the illustrated exemplary embodiment of FIG. 8A two air grooves 113a, 113b are illustrated. Preferably suction at each of the two air grooves 113a, 113b can be turned on and off separately, which would allow a surgeon to control the two air grooves 113a, 113b to create a situation wherein there is no suction, or a situation wherein there is suction at the first air groove 113a and/or at the second air groove 113b.

The first air groove 113a comprises a circular arc groove having a plurality of substantially radially extending arms. The circular arc groove surrounds the opening of the cutting element housing section 260 through which the cutting element extends at a distance thereof. The extending arms of the air groove 113a substantially enlarge the suction area of the first air groove 113a of the fixation portion. The circular arc groove fluidly interconnects the arms such that suction and/or vacuum is evenly distributed in the fixation portion 120. In a preferred embodiment, especially in view of exocone interventions, the first air groove 113a corresponds with a specimen area and may be controlled to provide suction, while the second air groove 113b may be controlled to not provide any suction, to allow a cut specimen to remain attached to the fixation portion 120 while removing the excision apparatus from the body.

A second air groove 113b forming a circular arc surrounding the first air groove is provided in the wall of the fixation portion 120. The circular arc of the second air groove has a larger diameter with respect to the circular arc of the first air groove 113a. The second air groove 113b allows to provide suction to obtain a vacuum to the outer most regions of the fixation portion 120 such that tissue situated near the edge 124 is sturdily retained by the fixation portion. In this way the fixation portion retains the tissue in an improved manner. The second air groove 113b comprises a groove portion which radially extends from the opening of the cutting element housing section 260 to the circular arc shaped portion of the second air groove. Said radially extending groove portion fluidly connects the circular arc shaped second air groove 113b to the air removal channels 113 or the cutting element housing section 260 and/or the electrode housing section 261.

It will be clear to those skilled in the art that a plurality of orientations, shapes and forms of the first and second air grooves are possible. FIG. 8B illustrates such a further exemplary embodiment where the first air groove 113a is circularly shaped to surround the opening of the cutting element housing section 260. The second air groove 113b comprises two circularly shaped portions where a first circularly shaped portion surrounds the second circularly shaped portion and is arranged near the edge 124 of the fixation portion 120. The second circularly shaped portion surrounds the first air groove 113b.

FIG. 9 is a perspective frontal view of the exemplary embodiment of the fixation portion illustrated in FIG. 8B.

FIG. 9 illustrates in particular a plurality of graduation indicators 125. The plurality of graduation indicators 125 are arranged and, preferably evenly, distributed near the circular arc of the second air groove 113b, i.e. near the periphery of the fixation portion 120. When the fixation portion 120 is arranged on, for example, a cervical area of a woman, the graduation indicators 125, in conjunction with a dial provided on the cutting element 130, provide the surgeon with visual feedback regarding the angular orientation of the cutting element 130 with respect to the position of the fixation portion 120. Such an embodiment is particularly useful as the surgeon would otherwise require the use of a medical imaging apparatus in order to visually follow his or her intervention or evenly rely entirely on personal muscular feedback from operating with the excision apparatus. The use of medical imaging apparatuses or relying on personal muscular feedback is prone to errors in performing the intervention and often results in medical complications with the patient or even infertility. Using the fixation portion 120 with such graduation indicators 125 provides the surgeon with visual feedback in a simple manner which reduces the risk for the patient and improves the patient's recovery after the intervention. According to an exemplary analogy, the graduation indicators 125 correspond to the numbers on a clock. According to said analogy, the graduation indicators at 12 h, 3 h, 6 h, 9 h would correspond to the limits of cutting positions I, II, III, IV of the four quadrants as described in FIG. 4B. It will be clear that a plurality of cutting position may be defined. For example, graduation indicators may be arranged such that they are angularly spaced apart wherein an angle between each position is for example 20°. In FIG. 9 it is apparent that the surgeon may insert the cutting element 130 via the cutting element housing section 260. The surgeon may rotate the cutting element in the retracted and the extended position of the cutting element 130. Rotating the cutting element 130 in the retracted position allows to position said cutting element in the correct starting position for performing the intervention. The surgeon may rotate the cutting element 130 to the required position indicated by a first graduation indicator 125. Moving the cutting element 130 to the extended position starts the intervention and cuts the tissue held by the fixation portion 120 along the axis A. Subsequently the surgeon may rotate the cutting element 130 to a second graduation indicator. Thus tissue is cut between the first and second graduation indicator. The surgeon may retract the cutting element 130 and/or retract the excision apparatus in its entirety to end the intervention. It will be clear that the cutting element may be rotated 360° with respect to the longitudinal axis A or a portion thereof. Preferably, and in addition to visually indicating the intended graduations of rotation, each of the graduation indicators 125 comprises a indentation or recess which provide tactile feedback to a surgeon to assist in cutting predefined sections and/or which create space for the cutting element 130, more in particular the electrode 140 thereof, to be efficiently and carefully placed and/or retracted at the corresponding location of the respective graduation indicator 125. It is clear for the skilled person that the graduation indicators 125 may be provided at a radius of the fixation portion 120 which corresponds best with the used electrode 140.

According to a preferred embodiment the excision apparatus is provided with a spring back mechanism (not illustrated) the spring back mechanism is configured to move the cutting element 130 from the extended position to the retracted position after the cutting element 130 reaches a predetermined rotational position. For example the spring back mechanism can move the cutting element when the cutting element rotates 360°. According to another example, the spring back mechanism moves the cutting element 130 after the cutting element has been rotated for 90°. The predetermined rotational position of the cutting element may correspond with the graduation indicators 125.

The embodiment illustrated in FIG. 9 is particularly useful for endocone interventions as the graduation indicators 125 in conjunction with a dial on the cutting element 130 provide visual feedback of the position of the cutting element 130 to the surgeon which would otherwise only be visible using medical imaging. In addition, the illustrated electrode 140 is especially suited for endocone interventions as the electrode comprises a transversal portion extending away from the cutting element 130 which is relatively short, and a longitudinal portion extending along the cutting element which is relatively long. The transversal portion is substantially perpendicular to the longitudinal axis A. Preferably the length of the transversal portion is between 1 mm and 4 mm. The longitudinal portion is substantially parallel to the longitudinal axis A. Preferably the length of the longitudinal portion is between 10 mm and 50 mm, more preferably between 15 mm and 45 mm, most preferably between 20 mm and 40 mm.

Although not shown in any of the previous figures, the excision apparatus may also be provided with a lighting means. The lighting means is configured to emit light. The lighting means improves the visibility of the surgeon, in particular during endocone or exocone interventions. The lighting means may be embodiment as an LED diode arranged on the housing 110. The lighting means may also be integrated in the housing, for example in the handling portion 230. The lighting means may for example be embodied as a lighting ring surrounding the handling portion such that the entire circumferential area of the excision apparatus is illuminated. The lighting means may also be directed to the intervention area, i.e.; the tissue being fixedly retained by the fixation portion. The lighting means may also be provided with optical guidance means which direct the emitted light from the lighting means to fixation portion thereby directly illuminating the tissue fixedly retained by the fixation portion or the tissue intended to be fixedly retained.

In the above described embodiments the cutting element is provided with an electrode to perform a cutting operation, however alternatively or in addition to the electrode, at least one of the following elements may be provided to perform the cutting operation: ultrasonic cutting means such as a harmonic scalpel, laser cutting means, mechanical cutting means. In addition, any one of the following elements may be provided to perform a sealing operation: ultrasonic sealing means, laser sealing means, mechanical sealing means.

Whilst the principles of the invention have been set out above in connection with specific embodiments, it is to be understood that this description is merely made by way of example and not as a limitation of the scope of protection which is determined by the appended claims.

1. An excision apparatus for removing cellular tissue, said excision apparatus comprising:
a housing provided with a fixation portion, wherein the fixation portion is configured to be arranged on cellular tissue such that a closed space is formed by the cellular tissue and an inner surface of the fixation portion; said fixation portion being further configured to fixedly retain the cellular tissue near the inner surface by removal of air from the closed space via an air evacuating means;
a cutting element moveably arranged in the housing such that the cutting element is movable between a retracted position and an extended position with respect to the fixation portion, wherein the cutting element comprises an electrode arranged at a distal end of the cutting element and configured for cutting a section of cellular tissue retained by the fixation portion when the cutting element is in the extended position.

2. The excision apparatus according to claim 1, wherein the cutting element is translatable along an axis with respect to the housing, wherein the axis is oriented according to a longitudinal direction of the housing.

3. The excision apparatus according to claim 1, wherein the fixation portion comprises at least one standing wall having an edge configured to come into contact with the cellular tissue and wherein in the extended position the cutting element does not extend beyond the edge of the fixation portion.

4. The excision apparatus according to claim 1, wherein the fixation portion is substantially cup-shaped.

5. The excision apparatus according to claim 1, wherein the cutting element is translatable over a distance measured along the axis of at least 3 mm.

6. The excision apparatus according to claim 1, wherein the cutting element is rotatable along the axis of the housing; and/or wherein the cutting element is rotatable between a plurality of indicated cutting positions, wherein each indicated cutting position corresponds with a respective section of tissue, and/or wherein the plurality of indicated cutting positions comprises four quadrants.

7-8. (canceled)

9. The excision apparatus according to claim 1, wherein at least the fixation portion is manufactured from a transparent material.

10. The excision apparatus according to claim 1, further comprising a guiding rod extending outwardly along the axis, in the direction of a distal end of the housing, and configured to be inserted in a body cavity, such as a cervix of a female patient, for aligning the excision apparatus with the walls of the body cavity.

11. The excision apparatus according to claim 1, wherein the electrode is a bipolar electrode comprising a first and a second subelectrode.

12. The excision apparatus according to claim 10, wherein the electrode forms the first subelectrode and wherein the guiding rod forms the second subelectrode.

13. The excision apparatus according to claim 11, wherein the electrode is a loop electrode; and/or wherein the loop electrode extends radially, seen in a projection on the axis, over a distance of at least 3 mm.

14. (canceled)

15. The excision apparatus according to claim 1, wherein the housing comprises an elongate intermediate portion, and wherein the fixation portion is arranged at a distal end of said elongate intermediate portion and is operably connected to said elongate intermediate portion such that air is removable from the closed space via the air evacuating means through the intermediate portion; and/or wherein the air evacuating means is formed by the elongate intermediate portion being spaced apart from and around the cutting element such that an air removal channel is formed and wherein the intermediate portion comprises an air evacuation interface being connectable to an air pump; and/or wherein one or more further air removal channels are formed by the elongate intermediate portion.

16-17. (canceled)

18. The excision apparatus according to claim 1, further comprising an actuating element being connected to the cutting element, wherein the actuating element is arranged at the proximal end of the housing and is configured for moving the cutting element between the retracted position and the extended position and/or for rotating the cutting element along the axis (A); and/or wherein the actuator element is arranged in an actuator housing section comprising an actuator housing wall, wherein the actuator housing wall comprises a plurality of position protrusions which are configured to be engaged by a position limiting element of the actuator element.

19. (canceled)

20. The excision apparatus according to claim 18, wherein the plurality of position protrusions define a plurality of axially oriented translation channels and tangentially oriented orientation channels; and/or the position limiting element slidingly engages the plurality of position protrusions such that the actuating element is moved and positioned with respect to the housing.

21. (canceled)

22. The excision apparatus according to claim 20, wherein the actuator housing wall is disposable on the outside thereof with indicators corresponding to the plurality of axially oriented translation channels and tangentially oriented orientation channels.

23. The excision apparatus according to claim 18, wherein an actuator housing section is provided at the proximal end of the housing and comprises a plurality of slots arranged side by side seen in a longitudinal direction along the axis; wherein the excision apparatus further comprises a position abutting element configured to prevent the cutting element from moving beyond the position abutting element, and wherein the position abutting element is arrangeable in a slot of the plurality of slots.

24. The excision apparatus according to claim 23, wherein the plurality of slots form a through hole and the position abutting element comprises an abutment which is intended to extend through the slot when the position abutting element is arranged on the actuator housing section and is configured to prevent the cutting element from moving beyond the abutment.

25. The excision apparatus according to claim 22, wherein the position abutting element is clippable on the housing, using a flexible or spring-loaded clip configured to engage the housing.

26. The excision apparatus according to any claim 1, wherein the fixation portion is configured to be releasably attachable to the distal end of the housing.

27. The excision apparatus according to claim 1, wherein the fixation portion is provided with one or more air grooves on the inner surface thereof, wherein the one or more air grooves extend over the inner surface; and wherein the fixation portion is provided with a plurality of graduation indicators, wherein the plurality of graduation indicators are evenly distributed seen along a circular arc on the fixation portion.

28. (canceled)
